(19) United States
(12) Patent Application Publication
(10) Pub. No.: US 2008/0138866 A1
(43) Pub. Date: Jun. 12, 2008

(54) Process for the Preparation of (R)-4,4-Dialkoxy-Pyran-3-Ols Such as (R)-4,4-Dimethoxy-Pyran-3-Ol

(76) Inventors: Jeffrey C. Moore, Westfield, NJ (US); Birgit Kosjek, Westfield, NJ (US); Joseph Nti-Gyabaah, Somerset, NJ (US)

Correspondence Address: MERCK AND CO., INC, P O BOX 2000, RAHWAY, NJ, 07065-0907, US

(21) Appl. No.: 11/885,309

(22) PCT Filed: Mar. 7, 2006
(86) PCT No.: PCT/US06/08133

§ 371 Date: Aug. 29, 2007

Related U.S. Application Data
(60) Provisional application No. 60/660,696, filed on Mar. 11, 2005.

(51) Int. Cl. C12P 17/06

(52) U.S. Cl. 435/125

(57) ABSTRACT

The present invention is concerned with novel processes for the preparation of (R)-4,4-dimethoxy-pyran-3-ol. This compound is useful as an intermediate in the synthesis of compounds which possess pharmacological activity including CCR2 antagonists.

### BACKGROUND OF THE INVENTION

Chiral compounds (R)-4,4-dialkoxy-pyran-3-ols, and in particular (R)-4,4-dimethoxy-pyran-3-ol, are important intermediates in the production of a useful class of therapeutic agents. However, the processes disclosed in the art for the preparation of (R)-4,4-dimethoxy-pyran-3-ol, and other (R)-4,4-dialkoxy-pyran-3-ols, result in relatively low and inconsistent yields of the desired product, and product having relatively low enantiopurity. Moreover, some of these processes rely on the use of expensive transition metal catalysts. As such, there is a need for the development of a process for the preparation of (R)-4,4-dimethoxy-pyran-3-ol, and other (R)-4,4-dialkoxy-pyran-3-ols, which is readily amenable to scale-up, avoids the use of costly transition metal catalysts, uses cost-effective and readily available reagents, and which is therefore capable of practical application to large scale manufacture.

In contrast to the previously known processes, the present invention provides effective methodology for the preparation of (R)-4,4-dimethoxy-pyran-3-ol, and other (R)-4,4-dialkoxy-pyran-3-ols, in relatively high yield and enantiomeric purity. Accordingly, the subject invention provides a process for the preparation of (R)-4,4-dimethoxy-pyran-3-ol, and other (R)-4,4-dialkoxy-pyran-3-ols, via a very simple, short and highly efficient synthesis.

### SUMMARY OF THE INVENTION

The present invention relates to an efficient and cost effective process for the preparation of (R)-4,4-dimethoxy-pyran-3-ol and other (R)-4,4-dialkoxy-pyran-3-ols. (R)-4,4-dimethoxy-pyran-3-ol is useful as an intermediate in the preparation of certain therapeutic agents. In particular, the present invention provides a process for the preparation of (R)-4,4-dimethoxy-pyran-3-ol. (R)-4,4-dimethoxy-pyran-3-ol is an intermediate in the synthesis of pharmaceutical compounds.

The novel process of this invention involves the synthesis of (R)-4,4-dialkoxy-pyran-3-ols:

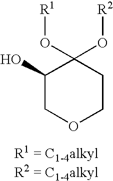

In particular, the present invention is concerned with novel processes for the preparation of the compound (R)-4,4-dimethoxy-pyran-3-ol of the formula:

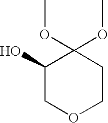

These compounds are intermediates in the synthesis of other compounds which possess pharmacological activity. In particular, these other compounds include but are not limited to CCR2 antagonists such as those described in WO03/092586, WO04/092124 and other publications. CCR2 antagonists are useful, e.g., in the treatment of inflammatory diseases and conditions, and in the treatment of other diseases and conditions.

### DETAILED DESCRIPTION OF THE INVENTION

The present invention is directed to processes for the preparation of (R)-4,4-dialkoxy-pyran-3-ols including the compound (R)-4,4-dimethoxy-pyran-3-ol of the formula:

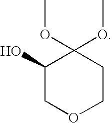

A preferred process for the preparation of (R)-4,4-dimethoxy-pyran-3-ol is described in the following scheme:

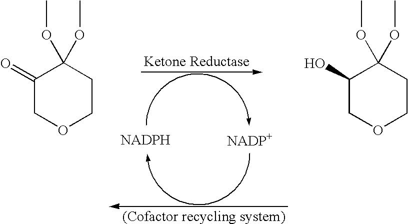

In accordance with this embodiment of the present invention, the treatment of 4,4-dimethoxy-pyran-3-one with a ketone reductase in the presence of nicotinamide adenine dinucleotide phosphate (NADPH), and a cofactor recycling system provides (R)-4,4-dimethoxy-pyran-3-ol in higher yields, in greater entantiomeric purity and in a more efficient route than the processes disclosed in the art.

Another embodiment of the general process for the preparation of (R)-4,4-dimethoxy-pyran-3-ol is described in the following scheme:

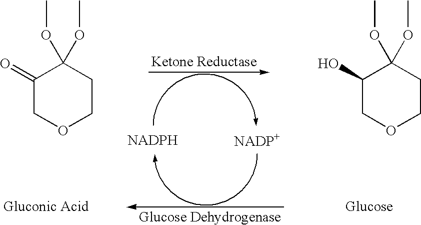

In accordance with this embodiment of the present invention, the treatment of 4,4-dimethoxy-pyran-3-one with a ketone reductase in the presence of nicotinamide adenine dinucleotide phosphate (NADPH) and a cofactor recycling system which comprises a glucose source and glucose coupled with a glucose dehydrogenase, provides (R)-4,4-dimethoxy-pyran-3-ol in higher yields, in greater entantiomeric purity and in a more efficient route than the processes disclosed in the art.

A further embodiment of the general process for the preparation of (R)-4,4-dimethoxy-pyran-3-ol is described in the following scheme:

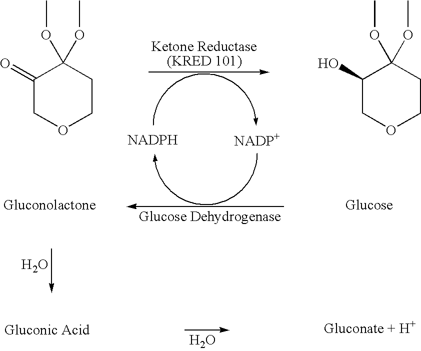

In accordance with this embodiment of the present invention, the treatment of 4,4-dimethoxy-pyran-3-one with a ketone reductase in the presence of nicotinamide adenine dinucleotide phosphate (NADPH), and a cofactor recycling system which comprises a glucose source and glucose coupled with a glucose dehydrogenase, provides (R)-4,4-dimethoxy-pyran-3-ol in higher yields, in greater entantiomeric purity and in a more efficient route than the processes disclosed in the art.

In another embodiment, the present invention is directed to a process for the preparation of (R)-4,4-dimethoxy-pyran-3-ol which comprises the treatment of 4,4-dimethoxy-pyran-3-one with a ketone reductase in the presence of NADPH, and a glucose source and a glucose dehydrogenase to give (R)-4,4-dimethoxy-pyran-3-ol.

A specific embodiment of the present invention concerns a process for the preparation of (R)-4,4-dimethoxy-pyran-3-ol of the formula:

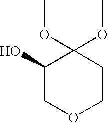

which comprises:

treating 4,4-dimethoxy-pyran-3-one of the formula:

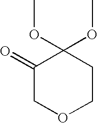

with a ketone reductase in the presence of nicotinamide adenine dinucleotide phosphate and a cofactor recycling system; to give (R)-4,4-dimethoxy-pyran-3-ol of the formula:

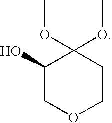

Other of (R)-4,4-dialkoxy-pyran-3-ols, for instance of (R)-4,4-diethoxy-pyran-3-ol, of (R)-4,4-dipropyloxy-pyran-3-ol and (R)-4,4-dibutyloxy-pyran-3-ols may be synthesized using analogous schemes.

In the present invention, the cofactor recycling system includes those which comprise glucose and glucose dehydrogenase, formate and formate dehydrogenase, glucose-6-phosphate and glucose-6-phosphate dehydrogenase, glucose-6-sulfate and glucose-6-phosphate dehydrogenase, alcohol and alcohol dehydrogenase. Other recycling methods useable in connection with the invention include electrochemical methods, photochemical methods, reducing agents, excess NADPH, or an alcohol as co-substrate for a coupled substrate approach.

In the present invention, the ketone reductase includes those selected from: Ketone REDuctase 101 (KRED101), Ketone REDuctase 102 (KRED102), Ketone REDuctase 105 (KRED105), Ketone REDuctase 107 (KRED107) and Ketone REDuctase 108 (KRED108), available commercially from Biocatalytics, Inc., and other ketone reductases.

In the present invention, the substrate 4,4-dimethoxy-pyran-3-one may be present at a concentration of about 95 to 105 g/L (0.69M to 0.66M). In a specific embodiment of the invention, the 4,4-dimethoxy-pyran-3-one may be present at a concentration of about 100 g/L (0.63M).

In the present invention, the ketone reductase may be present at a concentration of about 0.095 to 0.105 g/L {900 U to 1000 U (activity determined using 10 mM ethyl-4-chloroacetoacetate)}. In a specific embodiment of the invention, the ketone reductase may be present at a concentration of about 0.1 g/L {950 U (activity determined using 10 mM ethyl-4-chloroacetoacetate)}.

In the present invention, the nicotinamide adenine dinucleotide phosphate oxidized form (NADP+) may be present at a concentration of about 0.11 to 0.14 g/L (0.14 to 0.18 mM). In a specific embodiment of the invention, the nicotinamide adenine dinucleotide phosphate may be present at a concentration of about 0.12 g/L (0.15 mM).

In the present invention, the glucose source may be present at a concentration of about 120 to 140 g/L (0.66 to 0.77M).

In an embodiment of the present invention, the glucose dehydrogenase includes those selected from glucose dehydrogenase 101, glucose dehydrogenase 102, glucose dehydrogenase 103 (Biocatalytics) and mutants thereof, or glucose dehydrogenases from the following companies: Amano, Codexis, Sigma, and mutants thereof. In the present invention, the glucose dehydrogenase may be present at a concentration of about 0.28 to 0.33 g/L {5.6 to 6.6 MU (activity determined using 100 mM D-glucose)}. In a specific embodiment of the invention, the glucose dehydrogenase may be present at a concentration of about 0.3 g/L {6 MU (activity determined using 100 mM D-glucose)}.

In the present invention, the reaction mixture may comprise an aqueous buffer, such as a phosphate buffer. Those pH buffers useable in connection with the present invention include but are not limited to KH₂PO₄ and buffers of the range 6-8 such as MES, Bis-tris, PIPES, ACES, BES, MOPS, TES, HEPES and Tris. Thus, in an embodiment of the present invention, the pH of the reaction mixture is maintained between pH 6-8. In an aspect of this embodiment of the present invention, the pH of the reaction mixture is maintained at about pH 6.5. In another aspect of an embodiment of the present invention, the pH of the reaction mixture is maintained between pH 6-7, such as by the addition of an acid or base.

In the present invention, the reaction mixture may further comprise an solvent, such as methanol, ethanol, IPA, acetonitrile, DMSO. In an embodiment of the present invention, the solvent may be present at a concentration of no more than ˜10% v/v. In an embodiment of the present invention, the temperature of the reaction mixture is maintained at about 30 to 38° C. In a further embodiment of the present invention, the temperature of the reaction mixture is maintained at about 35° C.

For convenience, the ketone reductase, NADP, and a glucose source and a glucose dehydrogenase may be contacted together in situ, prior to reaction with 4,4-dimethoxy-pyran-3-one. Likewise for convenience, the ketone reductase, NADP, a glucose source and a glucose dehydrogenase, may be contacted together in situ, prior to reaction with 4,4-dimethoxy-pyran-3-one.

The (R)-4,4-dimethoxy-pyran-3-ol obtained in accordance with the present invention may be used as starting material in further reactions directly or following purification.

In a further embodiment, the present invention is directed to a process for purification of (R)-4,4-dimethoxy-pyran-3-ol which comprises: extracting the reaction mixture with a solvent selected from one or more of acetonitrile, toluene, alcohols (including but not limited to methanol, ethanol, propanol, butanol), methyl ethyl ketone, ethyl acetate, isopropyl acetate, and THF. The organic extract is then concentrated via vacuum distillation.

In an aspect of this further embodiment, extracting the reaction mixture with a solvent which comprises acetonitrile is conducted at a temperature of about 20 to 30° C.

In an alternate aspect of this further embodiment, the reaction mixture is saturated with 2M inorganic salt (such as NaCl, KCl), afterwhich the product is extracted with acetonitrile, and toluene is added to reduce the level of water in the organic extract. In an aspect of this further embodiment, concentrating the solvent is conducted by vacuum distillation at a jacket temperature of about 50-60° C.

It will be appreciated by those skilled in the art that extraction may be repeated in an iterative manner to further enhance the yield of (R)-4,4-dimethoxy-pyran-3-ol with each subsequent cycle.

Another aspect of this invention is directed (R)-4,4-dimethoxy-pyran-3-ol which is present in an enantiomeric purity (enantiomeric excess) of greater than 90%, greater than 95%, greater than 98%, greater than 99%, greater than 99.5% (enantiomeric excess) or greater than 99.9% (enantiomeric excess).

The starting materials and reagents for the subject processes are either commercially available or are known in the literature or may be prepared following literature methods described for analogous compounds. The skills required in carrying out the reaction and purification of the resulting reaction products are known to those in the art. Purification procedures include crystallization, distillation, normal phase or reverse phase chromatography.

The following examples are provided for the purpose of further illustration only and are not intended to be limitations on the disclosed invention.

### EXAMPLE 1

(R)-4,4-dimethoxy-pyran-3-ol: The following materials were prepared: 3.18 kg (2.9 L) aqueous dimethoxypyranone solution containing 0.62 kg dimethoxypyranone (3.88 moles), solution of 0.62 g KRED101 (5.9 MU) in 62 ml 0.5M phosphate buffer pH 6.5, solution of 1.86 g GDH (37.2 MU) in 62 ml 0.5M phosphate buffer pH 6.5, solution of 0.74 g NADP+ disodium salt (0.94 mmoles) in 62 ml 0.5M phosphate buffer pH 6.5 and solution of 0.8 kg glucose (4.48 moles) in 2 L 1.5M phosphate buffer at pH 6.5. The glucose solution was charged to a vessel and 3.18 kg aqueous dimethoxypyranone solution was added to give a final buffer concentration of 0.5M. The reaction was maintained at 35° C. The solutions of NADP+ and the two enzymes were added. Final reaction volume was 7.5 kg (6.2 L). The reaction was monitored by the pH drop, and stepwise adjustment of the pH from 6.0 to 6.5 was carried out by adding about 0.5 L 2.5M KHCO₃ solution every 2.5 hours. Completion of the reaction took place within 14 hours (100% AY, ee>98%). The pH was raised to 7.0 using 2.5M KHCO₃ to prepare for isolation. The resulting 9.7 L reaction mixture contains up to 620 g (R)-4,4-dimethoxy-pyran-3-ol.

### EXAMPLE 2

(R)-4,4-dimethoxy-pyran-3-ol: The following materials were prepared: 204 kg (193 L) aqueous dimethoxypyranone solution containing 38.3 kg dimethoxypyranone (239 moles), solution of 38.4 g KRED101 (365 MU) in 3.85 L 0.5M phosphate buffer pH 6.5, solution of 115.5 g GDH (2310 MU) in 3.85 L 0.5M phosphate buffer pH 6.5, solution of 47.6 g NADP+disodium salt (60 mmoles) in 3.85 L 0.5M phosphate buffer pH 6.5 and solution of 49.8 kg glucose (277 moles) in 32 L 1.5M phosphate buffer at pH 6.5. The glucose solution was charged to a vessel and 204 kg aqueous dimethoxypyranone solution was added to give a final buffer concentration of 0.5M. The reaction was maintained at 35° C. The solutions of NADP+ and the two enzymes were added. The reaction was monitored by the pH drop, and stepwise adjustment of the pH from 6.0 to 6.5 was carried out by adding about 83 L 2.5M KHCO₃ solution over the course of the reaction. Completion of the reaction took place within 18 hours (100% AY, ee>99%). The pH was raised to 7.0 using 2.5M KHCO₃ to prepare for isolation. The resulting 570 L reaction mixture contains up to 38.3 kg (R)-4,4-dimethoxy-pyran-3-ol.

### EXAMPLE 3

Extraction of (R)-4,4-dimethoxy-pyran-3-ol: 1.46 kg KCl (˜2M) was added to the reaction mixture from Example 1 (approximately 9.7 L containing up to 620 g (R)-4,4-dimethoxy-pyran-3-ol). Thereafter, 1.5 batch volumes (BV) of acetonitrile was added to extract (R)-4,4-dimethoxy-pyran-3-ol product, followed by the addition of 0.5 BV toluene to dry the organic layer. The organic and aqueous layers were cut into separate drums. The aqueous layer was then back extracted with a further 1.5 BV acetonitrile and 0.5 BV toluene. (FisherPak solvents used for all extractions.) The charges and volume distribution for the two extractions are summarized in the tables below:

Charges Made - First Extraction

| | Amount Charged (L) | Settling Time (min) |
|---|---|---|
| Acetonitrile | 14.6 | n/a |
| Toluene | 4.9 | ~20 |

Volume Distribution - First Extraction

| | Volume (L) |
|---|---|
| Organic Layer | 19.6 |
| Aqueous Layer | 8.7 |

Charges Made - Second Extraction

| | Amount Charged (L) | Settling Time (min) |
|---|---|---|
| Acetonitrile | 14.6 | n/a |
| Toluene | 4.9 | ~15 |

Volume Distribution - Second Extraction

| | Volume (L) |
|---|---|
| Organic Layer | 20.8 |
| Aqueous Layer | 8.3 |

### EXAMPLE 4

Vacuum Concentration Solvent Switch, (R)-4,4-dimethoxy-pyran-3-ol: Organic extracts (Example 3) were combined (40.4 L). Vacuum distillation under ˜28″ Hg vacuum, 55° C. bath, was performed until the volume reached ˜1 L (approximately 40-fold concentration). Thereafter, ˜10 L toluene was added. The concentrate was filtered to remove residual salts and the filter rinsed with ˜300 mL toluene. The flush was added to the original concentrate to give ˜1.32 final concentrate. The final concentrate was analyzed by GC to contain 449.3 g/L (R)-4,4-dimethoxy-pyran-3-ol product. Thus the yield for the isolation was 593.1 g, an overall yield of 95.6%.

### EXAMPLE 5

Extraction of (R)-4,4-dimethoxy-pyran-3-ol: 90.3 kg KCl was added to the reaction mixture from Example 2 (approximately 570 L containing up to ˜40 kg of (R)-4,4-dimethoxy-pyran-3-ol). Thereafter, 1.5 batch volumes (BV) of acetonitrile was added to extract (R)-4,4-dimethoxy-pyran-3-ol product, followed by the addition of 0.5 BV toluene to dry the organic layer. The organic and aqueous layers were cut into separate drums. The aqueous layer was then back extracted with a further 1.5 BV acetonitrile and 0.5 BV toluene.

### EXAMPLE 6

Vacuum Concentration, Solvent Switch, (R)-4,4-dimethoxy-pyran-3-ol: Organic extracts (Example 5) were combined, then vacuum distillation under ˜28″ Hg vacuum, 55° C. bath, was performed to concentrate the batch, after which toluene was added to complete the solvent switch into toluene. A total of 72.5 kg of R-dimethoxy alcohol solution in toluene was drummed off via a line filter into a PTFE lined drum. The contents of the drum were assayed was analyzed by GC to be 551 g/l and at a density of 1.009 kg/L. This equates to 39.6 kg of the R-dimethoxy alcohol. A total of 68.0 kg of R-dimethoxy alcohol solution in toluene was drummed off into a PTFE lined drum. The contents of the drum were analyzed by GC to be 551.5 g/l and at a density of 1.0198 kg/L. This equates to 39.5 kg of the R-dimethoxy alcohol.

### EXAMPLE 7

(R)-4,4-dipropyloxy-pyran-3-ol: The following materials were prepared: 50.5 g dipropyloxypyranone (0.23 moles), solution of 0.13 g KRED101 (1.2 MU) in 50 ml 0.5M phosphate buffer pH 6.5, solution of 0.39 g GDH (7.8 MU) in 13 ml 0.5M phosphate buffer pH 6.5, solution of 0.14 g NADP+ disodium salt (0.18 mmoles) in 35 ml 0.5M phosphate buffer pH 6.5 and solution of 80 g glucose (0.4 moles) in 200 ml DI water. The glucose solution was charged to a vessel and 50.5 g dipropylpyranone was added. The reaction was maintained at 30° C. The solutions of NADP+ and the two enzymes were added. Final reaction volume was 1 L. The reaction was monitored by the pH drop, and stepwise adjustment of the pH from 6.0 to 6.5 was carried out by adding about 100 ml 2.5M KHCO₃ solution over the course of the reaction. Completion of the reaction took place within 5 hours (100% AY, ee>98%). The resulting 1.1 L reaction mixture contains up to 50 g (R)-4,4-dipropyloxy-pyran-3-ol.

### EXAMPLE 8

Extraction of (R)-4,4-dipropyloxy-pyran-3-ol: A half batch volumes (BV) of acetonitrile, then another half batch volume of IPAc was added to the reaction mixture from Example 7 (1000 mL) to the extract (R)-4,4-dipropyloxy-pyran-3-ol product. The organic and aqueous layers were cut into separate bottles, afterwhich the aqueous layer was back extracted with a further 0.5 BV of acetonitrile and 0.5 BV of IPAc.

### EXAMPLE 9

Vacuum Concentration, (R)-4,4-dipropylox-pyran-3-ol: Organic extracts were combined, then vacuum distillation under ˜28″ Hg vacuum, 55° C. bath, was performed to concentrate the batch into oil. A total of 46 g of R-alcohol alcohol oil (analyzed to contain 45 g of product) was recovered.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, reaction conditions other than the particular conditions as set forth herein above may be applicable as a consequence of variations in the reagents or methodology to prepare the compounds from the processes of the invention indicated above. Likewise, the specific reactivity of starting materials may vary according to and depending upon the particular substituents present or the conditions of manufacture, and such expected variations or differences in the results are contemplated in accordance with the objects and practices of the present invention. It is intended, therefore, that the invention be defined by the scope of the claims which follow and that such claims be interpreted as broadly as is reasonable.

1. A process for the preparation of (R)-4,4-dialkoxy-pyran-3-ol of the formula: 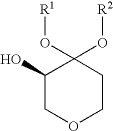
which comprises: reacting 4,4-dialkoxy-pyran-3-one of the formula: 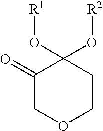
where R1 is independently C1-4alkyl, and where R2 is independently C₁-4alkyl, with a ketone reductase in the presence of nicotinamide adenine dinucleotide phosphate and a cofactor recycling system; to give a (R)-4,4-dialkoxy-pyran-3-ol of the formula:

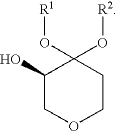

2. A process for the preparation of (R)-4,4-dimethoxy-pyran-3-ol of the formula: 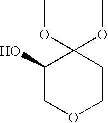
which comprises: reacting 4,4-dimethoxy-pyran-3-one of the formula: 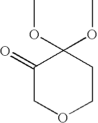
with a ketone reductase in the presence of nicotinamide adenine dinucleotide phosphate and a cofactor recycling system; to give (R)-4,4-dimethoxy-pyran-3-ol of the formula:

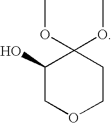

3. The process of claim 2 wherein said ketone reductase is selected from KRED101, KRED102, KRED105, KRED107 and KRED 108.

4. The process of claim 2 wherein the ketone reductase is KRED 101.

5. The process of claim 2 wherein the ketone reductase is present at a concentration of about 0.095 to 0.105 g/L.

6. The process of claim 2 wherein the ketone reductase is present at an activity of about 900 U to 1000 U.

7. The process of claim 2 wherein the substrate 4,4-dimethoxy-pyran-3-one is present at a concentration of about 95 to 105 g/L.

8. The process of claim 2 wherein the cofactor recycling system comprises glucose and a glucose dehydrogenase.

9. The process of claim 8 wherein the cofactor recycling system further comprises nicotinamide adenine dinucleotide phosphate.

10. The process of claim 9 wherein the nicotinamide adenine dinucleotide phosphate is present at a concentration of about 0.12 g/L.

11. The process of claim 8 wherein glucose is present at a concentration of about 120 to 140 g/L.

12. The process of claim 8 wherein the glucose dehydrogenase is present at a concentration of about 0.28 to 0.33 g/L.

13. The process of claim 2 wherein the reaction mixture comprises a phosphate buffer.

14. The process of claim 2 wherein the reaction mixture further comprises a solvent selected from methanol, ethanol, IPA, acetonitrile and DMSO.

15. The process of claim 2 comprising the further step of extracting the reaction mixture with a solvent selected from toluene, alcohol, acetonitrile, methyl ethyl ketone, ethyl acetate, isopropyl acetate, and THF.

16. The process of claim 15 wherein the reaction mixture is extracted with acetonitrile at a temperature of about 20° C. to 30° C.

17. The process of claim 15 wherein the reaction is extracted with acetonitrile, and wherein the organic layer is dried with toluene.

18. The process of claim 15 comprising the further step of concentrating the solvent by vacuum distillation.
